(19) United States
(12) Patent Application Publication
(10) Pub. No.: US 2025/0318949 A1
(43) Pub. Date: Oct. 16, 2025

(54) HANDS-FREE MENSTRUAL FLUID COLLECTION DEVICE WITH PRESSURE-ACTIVATED VALVES

(71) Applicants: Andreea Oana Mocanu, Easton, CT (US); Andrei Mircea Mocanu, Easton, CT (US); Radu Alexandru Mocanu, Easton, CT (US)

(72) Inventors: Andreea Oana Mocanu, Easton, CT (US); Andrei Mircea Mocanu, Easton, CT (US); Radu Alexandru Mocanu, Easton, CT (US)

(21) Appl. No.: 19/171,236

(22) Filed: Apr. 5, 2025

Related U.S. Application Data
(60) Provisional application No. 63/574,935, filed on Apr. 5, 2024.

(51) Int. Cl. A61F 5/455

(52) U.S. Cl. CPC A61F 5/4553

(57) ABSTRACT

A menstrual cup is disclosed, comprising a flexible, biocompatible collector configured for insertion into the vaginal canal and enabling hands-free emptying of menstrual fluid without removal. The collector body defines a fluid-retention chamber and includes one or more pressure-responsive valves and an outlet. The valves open in response to compression from voluntary abdominal or pelvic floor muscle contractions, allowing fluid evacuation while the device remains in situ. Upon release of pressure, the valves close to maintain a leak-proof seal. The valves also provide suction relief to equalize internal and external pressure during insertion or removal. Transverse reinforcement bands integrated into the collector body enhance pressure transfer and promote valve actuation. Unlike traditional menstrual cups that require removal for emptying, the present invention allows extended wear and periodic, hands-free drainage. The device may optionally incorporate biosensors and an applicator system for accurate placement of the cup into the vaginal canal.

### CROSS-REFERENCE TO RELATED APPLICATIONS

Provisional applications No. 63/574,935, filed on Apr. 5, 2024.

### FIELD OF THE INVENTION

The present invention relates generally to feminine hygiene products, and more specifically to intravaginal menstrual fluid collection devices that incorporate pressure-responsive valves for hands-free emptying.

This invention is classified under International Patent Class A61F 5/441 and A61F 5/455 for menstrual cups and bodily fluid collection devices. Further classifications include CPC A61F 5/4553 for vaginally inserted menstrual fluid collectors, and related classes A61F 6/06 and 6/12 for structural details.

### BACKGROUND OF THE INVENTION

Menstrual cups are commonly used as a reusable alternative to traditional absorbent menstrual products, such as tampons and sanitary pads. Typically fabricated from biocompatible materials including medical-grade silicone, rubber, or thermoplastic elastomers, these devices are inserted into the vaginal canal where they collect menstrual fluid. Their benefits-including higher fluid capacity, reduced environmental impact, and long-term cost-effectiveness-have contributed to increased consumer adoption.

Despite these advantages, conventional menstrual cups exhibit several usability limitations that hinder broader acceptance. A primary drawback is the requirement for periodic removal, manual emptying, and cleaning throughout the menstrual cycle. This removal process can be inconvenient, messy, and time-consuming, often resulting in spillage of collected fluid. In environments where access to clean water and private restroom facilities is limited-such as public restrooms, workplace settings, travel scenarios, or outdoor activities-maintenance of proper hygiene becomes particularly challenging. Many users report difficulty rinsing the cup discreetly when individual sinks, soap, or sanitizing supplies are unavailable.

Insertion and removal of existing menstrual cups can also be problematic. Proper use generally requires folding the cup prior to insertion, ensuring it unfolds and seals correctly against the vaginal walls to prevent leakage. First-time users frequently encounter difficulties with insertion or achieving a proper fit, which may lead to discomfort or ineffective fluid retention. Removal typically involves breaking the suction seal formed by the cup, a process that can cause pain or lead to unintended spillage if not performed correctly. These challenges are further exacerbated for individuals with anatomical variations or medical conditions such as vaginismus, a low cervix, or a retroverted uterus, reducing the accessibility of current menstrual cup designs.

Furthermore, individuals with heavy menstrual flow may be required to empty the cup significantly more often than the commonly cited 12-hour wear guideline. In such cases, menstrual cups may reach full capacity within 2 to 4 hours, necessitating frequent removal and reinsertion. This is particularly inconvenient in settings where clean restroom access is unavailable or time is constrained. Unlike disposable absorbent products, menstrual cups cannot be easily discarded or replaced; instead, they require a clean, private environment for hygienic handling. The need to manage full cups in public restrooms with communal sinks or limited privacy can be burdensome and deter use.

Nighttime use also presents difficulties for individuals with heavy flow, as it may necessitate waking during sleep to prevent overflow. This interrupts rest and undermines one of the principal benefits associated with menstrual cups-extended, uninterrupted wear time. While larger-capacity cup designs exist, they may still be insufficient for users with significantly high menstrual fluid volumes. As the frequency of emptying increases, so does the likelihood of fluid spillage during removal, especially when the cup is at or near capacity. This can be particularly problematic for inexperienced users or individuals with limited hand dexterity.

In scenarios where timely removal is not feasible-such as during long-haul travel, extended work shifts, athletic activities, or remote outdoor excursions-the risks of discomfort and leakage increase. Given these shortcomings, conventional menstrual cups fail to address the needs of individuals who require more frequent emptying and more discreet maintenance options.

Accordingly, there exists a need for a menstrual cup system that enables periodic emptying without the need for removal, thereby addressing key limitations of prior designs. A hands-free, in situ evacuation mechanism would significantly improve user convenience, reduce mess and spillage, and expand the suitability of menstrual cups for a broader range of users and use environments.

### SUMMARY OF THE INVENTION

A menstrual collector cup that empowers women to exert hands-free control over their menses flow, leveraging the natural contraction of vaginal muscles to facilitate effortless emptying of collected menses. The device comprises a flexible and resilient cup featuring an inner wall surface, an outer wall surface facing the vaginal wall, and one or more valves and outlet strategically designed to optimize fluid dynamics. The top proximal end of the collector has an upper rim circular edge section that expands and seals to maintain a pre-selected position within the vagina, ensuring a snug and secure fit. The main section extends downwards from this rim towards the distal bottom end, where one or more embedded mini-valves are integrated. This valve responds to voluntary pressure induced by the contraction of the human user's diaphragm muscle, opening to facilitate effortless emptying of accumulated menses volume and subsequently closing when the pressure is released. Additionally, the valve affords a vacuum relief function that equalizes air pressure inside and outside the cup for easier removal. In various embodiments, the distal outlet structure may include a single valve or two or more valves disposed sequentially in a downstream configuration depending on the intended fluid control profile. This menstrual collector offers numerous benefits, including hands-free control over menses flow, reduced mess and hassle compared to traditional menstrual cups. The device's embedded mini-valve prevents leakage and enables smooth menstrual flow in emptying the cup without necessitating its removal or manual compression of the valve. The device also includes an applicator which easily inserts and positions the device into the vagina ensuring a high level of comfort and ease-of-use. Overall, this invention presents a groundbreaking approach to menstrual collection and management, providing a convenient, hygienic, and empowering solution for women.

### DETAILED DESCRIPTION OF THE INVENTION

The present invention relates to a menstrual fluid collection device configured for insertion into the vaginal canal. More specifically, the invention enables hands-free, in situ emptying of collected menstrual fluid by utilizing voluntary muscular contractions-such as diaphragm-induced intra-abdominal pressure combined with pelvic floor relaxation-to actuate outlet valves. The invention addresses key drawbacks of conventional menstrual cups, which require manual removal and external handling for emptying, by offering a cleaner, more discreet, and extended-wear solution.

The device includes a flexible, biocompatible collector section that defines a fluid-retention cavity, and a distal outlet structure incorporating one or more valves that open and close automatically in response to user-generated pressure. In some embodiments, the collector wall includes transverse striae bands that assist in distributing compression force and promoting efficient fluid expulsion through the outlet. An optional applicator assembly may be used to facilitate hygienic insertion of the device.

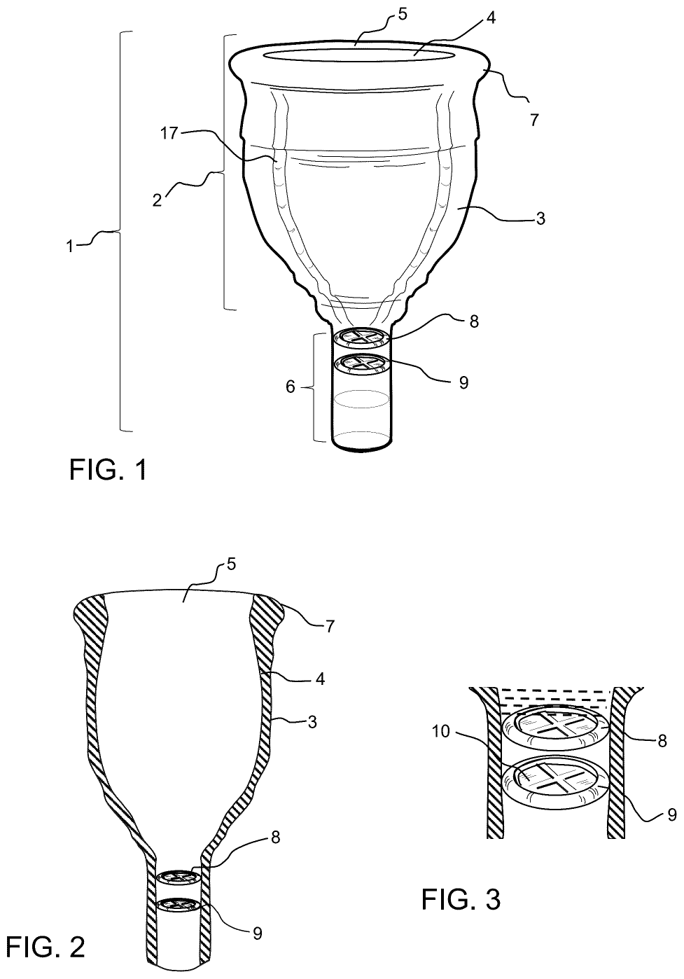

Referring now to FIGS. 1-9, the invention will be described in detail through illustrative embodiments.

As shown in FIGS. 1-2, the menstrual fluid collection device (1) includes a collector section (2) defined by a wall (3) having an inner surface (4), which defines a central cavity adapted to collect menstrual fluid, and an outer surface, which is in contact with the surrounding vaginal tissue when deployed. The collector section (2) extends longitudinally from an open top end (5) to a distal funnel tube (6).

The open top end (5) of the collector section (2) has a predetermined outside diameter sized for sealing contact with the vaginal canal. A thickened upper rim (7) is formed at the top end (5) of the wall (3), providing radial stiffness and assisting in maintaining the device in a pre-selected, stable position once inserted. The rim (7) helps ensure leak prevention and user comfort during use.

The wall (3) of the collector section (2) is made of a flexible and resilient biocompatible material, such as medical-grade silicone. Its thickness and elasticity vary along its length, with the wall tapering from the upper rim (7) to the lower region near the funnel tube (6). Integrated into the wall (3) are one or more transversal striae bands (17), which are composed of thicker material than the surrounding regions. These bands (17) enhance structural integrity and promote internal pressure buildup when the user applies voluntary abdominal or pelvic pressure (e.g., by bearing down). This deformation of the collector wall assists in initiating flow through the outlet.

As shown in FIGS. 3-7, the funnel tube (6) serves as a discharge pathway and houses at least one valve, and in the illustrated embodiment, two valves: a first valve (8) positioned proximally in the funnel tube and a second valve (9) located at the distal tip. The valves (8, 9) are preferably cross-slit (X-type) valves, although other elastomeric valve designs may be used. Each valve includes cusps or flaps (10) that remain closed under normal pressure and open when sufficient fluid or muscular force is applied from within the collector.

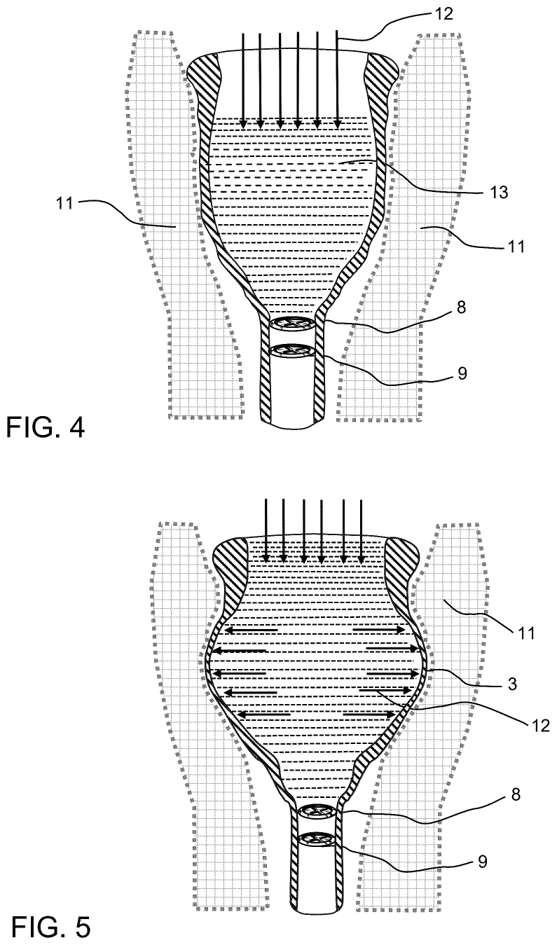

During use, menstrual fluid (inflow 12) enters the collector cavity through the open top end (5) while the valves (8, 9) remain closed, as shown in FIG. 4. As fluid accumulates, it exerts pressure against the inner surface (4) of the wall (3), which in turn applies outward pressure against the vaginal wall and internal muscles, resulting in a sensation of fullness or abdominal pressure, as seen in FIG. 5.

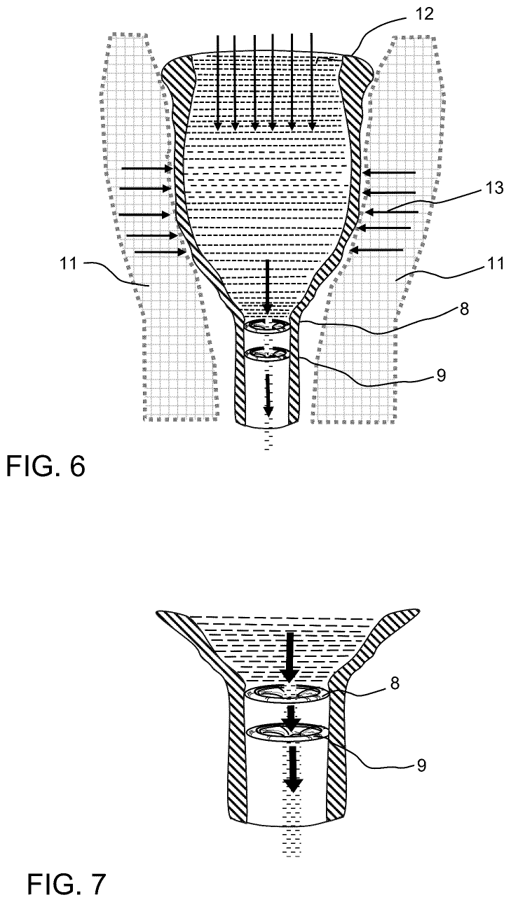

When the user voluntarily bears down-mimicking the physiological action involved in defecation or urination-the resulting intra-abdominal pressure, combined with pelvic floor relaxation, compresses the collector wall (3), particularly at the location of the striae bands (17), as shown in FIG. 6. This force promotes the opening of the first valve (8), followed by the second valve (9), allowing menstrual fluid to be expelled through the funnel tube (6) in a controlled, hands-free manner. The use of bearing-down maneuvers to increase pelvic pressure is well-documented in clinical practice and pelvic floor therapy, including in Rao et al., “Treating pelvic floor disorders of defecation: Management or cure?”, Current Gastroenterology Reports, vol. 11, pp. 278-287, 2009. This reference supports the effectiveness of the described physiologic action in creating pressure sufficient to actuate passive valves.

Once the pressure is released, the valves (8, 9) automatically return to their closed position due to the resilience of the valve material, maintaining a seal to prevent leakage or backflow. The second valve (9) acts as a secondary barrier and assists in smoothing the flow path during discharge. In alternative embodiments, only a single valve may be used for users with lighter menstrual flow.

In addition to regulating fluid outflow, the valve assembly—especially the first valve (8)—also serves a vacuum relief function. During insertion or removal of the device, the valve permits two-way airflow at a predetermined pressure differential, equalizing internal and external pressures. This feature reduces discomfort and prevents the formation of a vacuum seal, easing both deployment and extraction of the device.

When the user desires to remove the device (1), they may manually grasp the lower portion of the funnel tube (6) and gently withdraw the device. Preferably, the device is emptied through the valves prior to removal, minimizing mess and improving user experience.

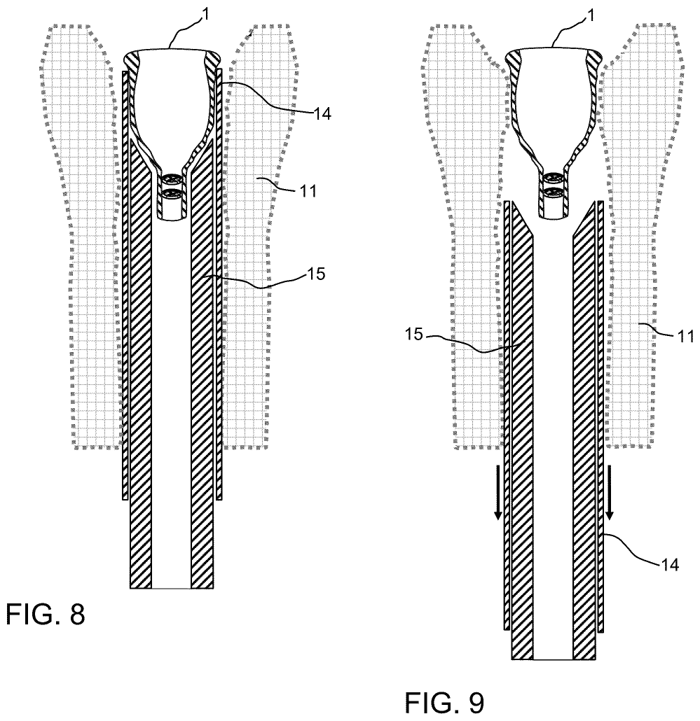

To facilitate insertion, the invention may further comprise an applicator assembly, as illustrated in FIGS. 8-9. The applicator includes an outer barrel tube (14) and an inner pusher tube (15). The menstrual cup (1) is folded and loaded into the barrel tube (14), which has a rounded insertion end. The pusher tube (15) is sized to slide within the barrel tube (14) and is approximately twice its length. During use, the loaded barrel tube (14) is inserted into the vagina, and the pusher (15) is advanced to expel the menstrual cup (1) into the vaginal cavity. The cup then expands and its upper rim (7) engages the vaginal walls to form a stable seal. The applicator is then withdrawn.

The applicator system supports controlled, hygienic, and precise placement, reducing user contact with bodily fluids and minimizing discomfort. This is particularly beneficial for first-time users or those with dexterity limitations.

The menstrual fluid collection device (1), including the collector section (2), funnel tube (6), valves (8, 9), and applicator components (14, 15), may be manufactured from medical-grade silicone, thermoplastic elastomers, or other biocompatible materials suitable for prolonged internal use. Optional embodiments may include antimicrobial coatings or biosensors for monitoring menstrual health or fluid characteristics.

In certain embodiments, the menstrual fluid collection device may further include one or more biosensors embedded in or affixed to the wall of the collector section (2) or the inner surface (4). These biosensors may be configured to monitor menstrual health parameters such as fluid volume, temperature, pH, hormone concentrations, or fluid viscosity. The biosensors may collect data passively or in real time and may optionally be configured to transmit data wirelessly to an external receiver, mobile device, or digital health platform. The inclusion of biosensors does not alter the passive valve-based fluid evacuation mechanism and may be implemented independently of the primary fluid discharge function.

Although specific embodiments have been described, the invention is not limited thereto. Modifications, substitutions, and equivalents may be made by those skilled in the art without departing from the scope of the invention as defined in the appended claims. It is understood that certain dependent claims, such as those directed to specific valve configurations (e.g., cross-slit valves with flaps) or particular applicator types, represent preferred embodiments and are not intended to limit the broader scope of the invention as defined in the independent claims. These specific features serve as illustrative examples of suitable implementations and are provided to support potential fallback positions without restricting coverage to only those variations.

### BRIEF DESCRIPTION OF THE DRAWINGS

Referring now to the figures, a preferred embodiment of a menstrual fluid collection device in accordance with the present invention is shown and described. As illustrated in FIGS. 1-9, the device (1) is designed for insertion into the vaginal canal and configured to collect and periodically discharge menstrual fluid without removal from the body.

The device (1) comprises a collector section (2) having a flexible wall (3) with an inner surface (4) that defines a collection cavity adapted for the accumulation of menstrual fluid, and an opposed outer surface that contacts the vaginal wall. The collector section (2) extends longitudinally from an open top end (5) to a distal funnel tube (6), which terminates in one or more outlet valves.

The open top end (5) has a predetermined outer diameter and is designed to maintain the device (1) in a preselected position within the vagina. A reinforced upper rim (7) forms part of the wall (3) at the proximal end and serves to expand and stabilize the collector section (2) during deployment and use. The upper rim (7) is the thickest region of the wall (3), ensuring structural support to maintain an effective seal against the vaginal walls.

The wall (3) of the collector section (2) is manufactured from a biocompatible, flexible, and resilient material such as medical-grade silicone. The wall (3) may exhibit variable thickness, decreasing from the upper rim (7) toward the distal funnel tube (6), thereby providing comfort and flexibility during use. Integrated into the wall (3) are one or more transversal striae bands (17), formed from a thicker portion of the same material. These bands (17) are oriented transversely around the circumference of the collector section (2) and are designed to improve pressure transfer and controlled deformation of the device. During voluntary abdominal or pelvic contractions-such as bearing down-these striae bands (17) facilitate internal pressure increase within the collector cavity and aid in the activation of the outlet valves for fluid expulsion.

The distal funnel tube (6) extends from the lower end of the collector section (2) and houses one or a plurality of valves. As illustrated in FIGS. 3 and 6-7, the preferred embodiment includes a first valve (8) embedded proximally within the funnel tube (6), and a second valve (9) embedded at the lower distal end. The valves (8, 9) are preferably cross-slit or “X”-type valves and may include flexible flaps or cusps (10) that open in response to internal fluid pressure. The design allows for non-tortuous, direct flow paths suitable even for viscous menstrual fluid.

In operation, and as shown in FIGS. 4-6, the collector section (2) fills with menstrual fluid (inflow 12) while the valves (8, 9) remain closed under normal conditions. As fluid accumulates, pressure builds within the cavity. When the user performs a bearing-down contraction (13)-similar to actions involved in defecation or urination-the increased intra-abdominal pressure and coordinated pelvic floor muscle relaxation create compression on the wall (3) and activate the striae bands (17), resulting in fluid expulsion through the opened valves (8, 9) via the funnel tube (6). Once the pressure is released, the valves automatically return to a closed position to prevent leakage.

Importantly, this mechanism allows for hands-free, in situ emptying of the collector section (2) without the need for manual squeezing, external valve manipulation, or removal of the device (1). This is particularly beneficial in public settings, during travel, or in situations where access to private hygiene facilities is limited.

Additionally, at least one of the valves (preferably the first valve (8)) is configured to provide a vacuum-relief function, permitting air to enter the collector cavity under defined pressure differentials. This facilitates easier insertion and removal of the device (1) by minimizing suction forces. As shown in FIG. 7, once the valves (8, 9) open under user-induced pressure, a free-flow of menstrual fluid is permitted through the funnel tube (6) and out of the body.

When the user wishes to remove the device (1), as depicted in FIG. 8, the lower portion of the device-primarily the distal funnel tube (6)-may be gently grasped and withdrawn. In preferred use, the user initiates fluid evacuation via muscle contractions before removal, resulting in an empty collector section (2) and a more comfortable and hygienic removal process.

To address insertion challenges common in conventional menstrual cup use, the device (1) may be deployed using an insertion assembly comprising concentric tubes (14 and 15), as shown in FIGS. 8 and 9. The folded menstrual cup is preloaded into an outer barrel tube (14), which receives a slidable inner pusher tube (15). During insertion, the barrel tube (14) is introduced into the vaginal canal and the pusher tube (15) is advanced, expelling the device (1) into the vagina. The collector section (2) then expands, and the upper rim (7) forms a seal against the vaginal walls. The applicator is withdrawn, leaving the device (1) correctly positioned.

This applicator system promotes hygienic, accurate placement and reduces user discomfort, particularly for first-time users or individuals with anatomical variability. The collector body and valves may also be optionally coated with antimicrobial material or embedded with biosensors to monitor menstrual health parameters.

All embodiments described are intended to be illustrative and not limiting. Various modifications may be made within the scope of the invention, as defined in the claims.

1. A menstrual fluid collection device comprising:
a flexible collector body having a proximal open end and a distal end, the collector body defining a fluid-retention cavity;
a rim disposed at the proximal open end, the rim configured to engage the vaginal wall to seal the collector body in place and prevent fluid leakage; and
a distal outlet structure located at the distal end of the collector body, the distal outlet structure comprising at least one valve,
each valve comprising one or more deformable regions configured (i) to transition from a closed state to an open state in response to pressure voluntarily applied by abdominal or pelvic floor muscle contractions to allow fluid evacuation from the fluid-retention cavity, and (ii) to return to the closed state upon release of pressure,
the at least one valve being further configured to permit two-way airflow to equalize internal and external pressure during insertion or removal of the device.

2. The menstrual fluid collection device of claim 1, wherein the collector body has a longitudinal axis and comprises a tubular wall extending from the proximal open end to the distal end.

3. The menstrual fluid collection device of claim 1, wherein the rim is thickened relative to the collector body and provides structural reinforcement to maintain the device in a preselected position within the vaginal canal.

4. The menstrual fluid collection device of claim 1, wherein the collector body includes one or more transverse bands formed of thickened material, the bands being configured to enhance wall deformation and internal pressure transmission during voluntary muscle contractions.

5. The menstrual fluid collection device of claim 1, wherein the valves are cross-slit valves composed of an elastomeric material and include flaps that open in response to internal pressure.

6. The menstrual fluid collection device of claim 1, wherein the valves are configured to allow hands-free evacuation of menstrual fluid while the collector body remains positioned within the vaginal canal.

7. The menstrual fluid collection device of claim 1, wherein each valve comprises one or more deformable regions configured to transition from a closed state to an open state in response to internal pressure and to return to the closed state upon pressure release.

8. The menstrual fluid collection device of claim 1, wherein the distal outlet structure comprises a first valve and a second valve arranged in series.

9. The menstrual fluid collection device of claim 1, wherein the collector body is formed from an elastomeric material selected from the group consisting of: medical-grade silicone, thermoplastic elastomer, polyurethane, or other biocompatible polymers.

10. The menstrual fluid collection device of claim 1, wherein the valve is configured to open in response to intra-abdominal pressure, including pressure generated during urination, allowing concurrent evacuation of menstrual fluid.

11. A method of evacuating menstrual fluid from a menstrual fluid collection device positioned within the vaginal canal, the method comprising:
(a) providing a menstrual fluid collection device comprising:
(i) a flexible collector body having a proximal open end and a distal end, the collector body defining a fluid-retention cavity;
(ii) a rim at the proximal open end configured to engage the vaginal wall and maintain the device in position; and
(iii) a distal outlet structure comprising at least one valve configured to open in response to internally applied pressure and to close upon release of said pressure;
(b) collecting menstrual fluid within the fluid-retention cavity while the device remains in situ within the vaginal canal;
(c) voluntarily contracting abdominal or pelvic floor muscles to generate intra-abdominal pressure; and
(d) opening at least one valve by the applied pressure to evacuate menstrual fluid through the distal outlet structure without removal of the device.

12. The method of claim 11, wherein the valve is a cross-slit elastomeric valve comprising a plurality of flexible flaps.

13. The method of claim 11, wherein the evacuation of menstrual fluid occurs simultaneously with urination.

14. The method of claim 11, further comprising allowing the valve to function as a vacuum-relief mechanism during removal of the device.

15. The method of claim 11, further comprising inserting the menstrual fluid collection device into the vaginal canal using an applicator comprising:
i. a barrel tube having a proximal end shaped for vaginal insertion; and
ii. a pusher slidably disposed within the barrel tube and configured to advance the device from the barrel tube into the vaginal canal.

16. The method of claim 11, wherein the menstrual fluid collection device comprises a first valve and a second valve arranged in series within the distal outlet structure, and both valves are sequentially actuated by the applied pressure to permit fluid evacuation.

17. The method of claim 11, wherein the collector body comprises one or more transverse bands of thickened material configured to enhance pressure transfer to the valve during abdominal or pelvic floor muscle contraction.

18. The method of claim 11, further comprising monitoring one or more physiological parameters using one or more biosensors disposed on or within the collector body.

19. The method of claim 18, wherein the physiological parameters comprise one or more of:
fluid volume, pH, temperature, hormone concentration, or viscosity.

20. The method of claim 18, further comprising transmitting biosensor data to an external receiver or software application during or after fluid collection.
